(19) United States
(12) Patent Application Publication
(10) Pub. No.: US 2024/0206713 A1
(43) Pub. Date: Jun. 27, 2024

(54) NEUTRAL ALIGNMENT INDICATOR FOR AN ACTUATOR OF AN INSERTION TUBE OF AN ENDOSCOPIC DEVICE

(71) Applicant: Bhavesh B. Shah, Cleveland, OH (US)

(72) Inventor: Bhavesh B. SHAH, Cleveland, OH (US)

(21) Appl. No.: 18/529,519

(22) Filed: Dec. 5, 2023

Related U.S. Application Data
(60) Provisional application No. 63/435,406, filed on Dec. 27, 2022.

(51) Int. Cl. A61B 1/005

(52) U.S. Cl. CPC A61B 1/0052; A61B 2090/0808

(57) ABSTRACT

An endoscopic device comprises a control portion including a housing and an actuator. An insertion tube is configured for insertion into a cavity of a patient to perform at least one of visual inspection and a therapeutic intervention. The actuator is configured to articulate an articulating end of the insertion tube. A first alignment indicator located on the housing. A second alignment indicator located on the actuator.

When the second alignment indicator on the actuator is aligned with the first alignment indicator on the housing, the insertion tube is articulated in a neutral position allowing insertion and removal of the insertion tube from the cavity of the patient.

### CROSS-REFERENCE TO RELATED APPLICATIONS

This application claims the benefit of U.S. Provisional Application No. 63/435,406, filed on Dec. 27, 2022. The entire disclosure of the application referenced above is incorporated herein by reference.

### FIELD

The present disclosure relates to endoscopic devices, and more particularly to a neutral alignment indicator for an actuator of an insertion tube of an endoscopic device.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Endoscopic devices are utilized in gastroenterology, pulmonology, otolaryngology, and/or urology procedures for various reasons including observation and/or therapeutic intervention. An operator of the endoscopic device manipulates control actuators such as rotary dials, buttons, and/or levers to perform visual inspection and/or therapeutic intervention. The endoscopic devices are manufactured in both reusable and disposable single-use formats.

Although uncommon, complications may occur when performing procedures utilizing endoscopic devices. For example, perforation of a viscous organ or trauma related to the insertion or removal of the endoscopic device are known risks of the procedure. Endoscopic devices manufactured in a reusable format or in a single-use disposable format have different actuator characteristics including endoscopic dial tension, tactile feel and/or recoil.

### SUMMARY

An endoscopic device comprises a control portion including a housing and an actuator. An insertion tube is configured for insertion into a cavity of a patient to perform at least one of visual inspection and a therapeutic intervention. The actuator is configured to articulate an articulating end of the insertion tube. A first alignment indicator located on the housing. A second alignment indicator located on the actuator. When the second alignment indicator on the actuator is aligned with the first alignment indicator on the housing, the insertion tube is articulated in a neutral position allowing insertion and removal of the insertion tube from the cavity of the patient.

In other features, the actuator includes a first articulation knob and a second articulation knob that are mounted coaxially. The first articulation knob includes the second alignment indicator. The second articulation knob includes a third alignment indicator.

In other features, the insertion tube is in the neutral position when the second alignment indicator on the first articulation knob and the third alignment indicator on the second articulation knob are aligned with the first alignment indicator on the housing.

In other features, the actuator includes a first articulation knob and a second articulation knob. The first articulation knob includes the second alignment indicator and a third alignment indicator. One of the housing and the second articulation knob includes a fourth alignment indicator.

In other features, the insertion tube is in the neutral position when the second alignment indicator on the first articulation knob is aligned with the first alignment indicator on the housing and the third alignment indicator on the first articulation knob is aligned with the fourth alignment indicator on the second articulation knob.

In other features, the first alignment indicator and the second alignment indicator are visually different than the third alignment indicator and the fourth alignment indicator. The actuator comprises a lever.

Further areas of applicability of the present disclosure will become apparent from the detailed description, the claims, and the drawings. The detailed description and specific examples are intended for purposes of illustration only and are not intended to limit the endoscopic device of the disclosure.

### BRIEF DESCRIPTION OF THE DRAWINGS

The present disclosure will become more fully understood from the detailed description and the accompanying drawings, wherein:

FIG. 1 is a simplified, side view of an example of an endoscopic device according to the present disclosure;

FIG. 2A is an enlarged, perspective view of an example of the control portion of the endoscopic device including a housing and actuators with alignment indicia to enable alignment of the actuators in a neutral position;

FIG. 2B is an enlarged perspective view of an example of an articulation knob of the endoscopic device of FIG. 2A;

FIG. 3 is an enlarged, perspective view of another example of the control portion of the endoscopic device including a housing and actuators with alignment indicia to enable alignment of the actuators in a neutral position; and

FIG. 4 is a functional block diagram of an example of an endoscopic device including an actuator with an alignment indicator according to a present disclosure.

In the drawings, reference numbers may be reused to identify similar and/or identical elements.

### DETAILED DESCRIPTION

Endoscopic devices include a control portion that is held by an operator during a procedure and an insertion tube extending from the control portion that is inserted into a cavity of a patient. The control portion includes a housing and one or more actuators such as articulation knobs, articulation levers, buttons and/or other actuators that are mounted on the housing and moveable relative to the housing. The insertion tube is inserted into a cavity of the patient during the procedure and later removed from the patient after the procedure. During the procedure, the actuators of the control portion are used to articulate the end of the insertion tube.

Articulation of the insertion tube of the endoscopic device is controlled solely by the actuators. For patient safety, the insertion tube of the endoscopic device should be returned to a neutral position corresponding to a non-tensioned, relaxed, unbiased, and/or natural position during certain maneuvers (e.g., insertion or removal of the endoscopic device from the patient). For example, failure to return the actuators of the endoscopic device to the neutral position prior to removal of the insertion tube may result in patient injury.

Actuators do not necessarily return to the neutral position automatically when released by the operator. Therefore, the operator needs to manually return the actuator controls to the neutral position before insertion and/or removal. However, endoscopic devices are manufactured by multiple manufacturers and have both reusable formats and a single-use disposable formats. The endoscopic devices have different layouts and actuator characteristics (e.g., endoscopic dial tension, tactile feel and/or recoil). Therefore, it is not always readily apparent to the operator when the endoscopic device is in the neutral position. Furthermore, it may not be clear from visual inspection that the actuators of the endoscopic device are in the neutral position.

Endoscopic devices according to the present disclosure include a control portion and an insertion tube extending from the control portion. The insertion tube is inserted into the patient during the procedure, articulated, and eventually removed from the patient after the procedure. The control portion includes a housing and one or more actuators that are mounted on and moveable relative to the housing. The operator manipulates the one or more actuators during the procedure to perform the observation and/or therapeutic intervention.

The one or more actuators are configured to articulate the insertion tube relative to the control portion from a neutral position to one or more other articulated positions. In some examples, the insertion tube is in a straight line when in the neutral position (or another position). In some examples, the housing of the control portion and the one or more actuators include alignment indicators identifying the neutral position of the endoscopic device. When the alignment indicators on the one or more actuator controls are aligned with the alignment indicator on the housing, the operator can safely insert or remove the insertion tube from the patient.

In operation, the operator ensures that the alignment indicators on the actuators are aligned with the indicators on the housing prior to inserting the insertion tube into the cavity of the patient. The operator performs the observation and/or therapeutic intervention. When the observation and/or therapeutic intervention is complete, the operator ensures that the alignment indicators on the actuators are aligned with the indicators on the housing to ensure that the insertion tube is in the neutral position prior to removing the insertion tube from the patient. As a result, the risk of perforation of a viscous organ and/or trauma related to the insertion and/or removal of the endoscopic device is significantly reduced.

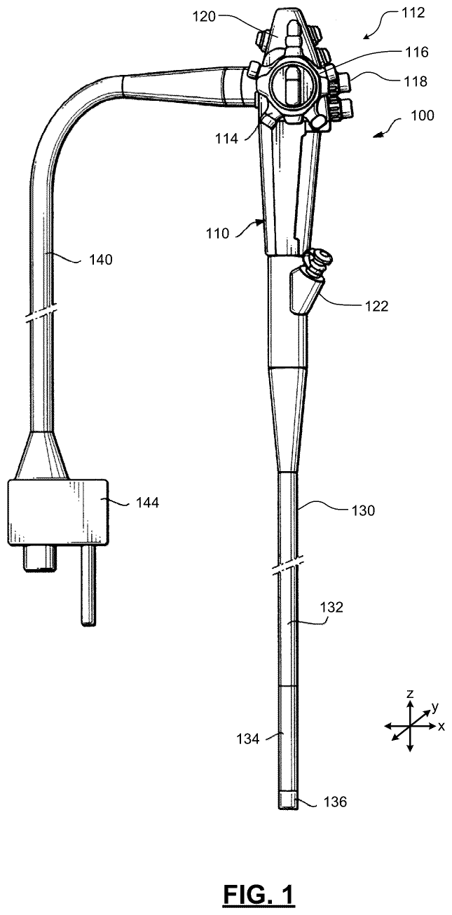

Referring now to FIG. 1, an endoscopic device 100 such as a bronchoscope, cystoscope, duodenoscope, gastroscope, rhinoscope, etc. includes a control portion 110 including a housing 120 and actuators 112. The actuators 112 include one or more articulation knobs 114 and 116, buttons 118 and/or other controls that are moveable relative to the housing 120. In some examples, the articulation knobs 114 and 116 include circumferentially spaced, radial projections 115 and 117, respectively.

One end of an insertion tube 130 is connected to the control portion 110. The insertion tube 130 is configured to be inserted into a cavity of a patient during a procedure to perform visual inspection and/or therapeutic intervention. The insertion tube 130 includes an articulating portion 134 hear the distal end thereof. Some of the actuators 112 (such as the one or more articulation knobs 114 and 116) are configured to cause articulation of the articulating portion 134 of the insertion tube 130 in x, y and/or z planes. An end 136 of the insertion tube 130 includes one or more devices such as a light guide to supply light, an air/water nozzle to supply air or water, a lens to allow images to be taken, a biopsy port, and/or other devices.

A connector 140 is used to connect the control portion 110 to an interface for connecting a video controller and display (not shown) and/or a light source (not shown). The light source supplies light to the end 136 of the insertion tube 130 to illuminate objects. The video controller and display processes image signals from the lens. In some examples, optical fiber is used to supply light to the end 136 of the insertion tube 130 and to connect the lens to the video controller and display.

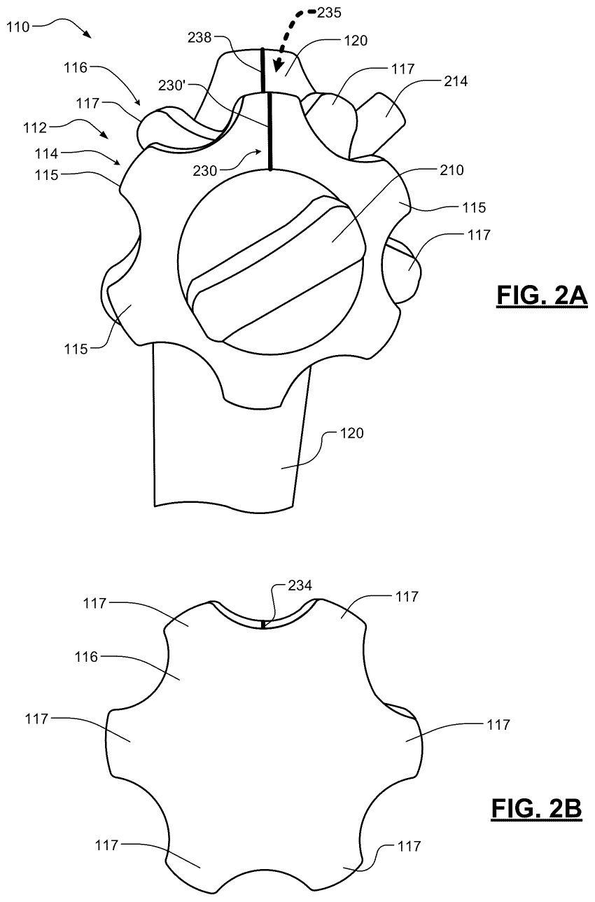

Referring now to FIGS. 2A and 2B, an enlarged, perspective view of the control portion 110 of the endoscopic device is shown. The actuators 112 are shown to include the articulation knobs 114 and 116. In some examples, the articulation knobs 114 and 116 are coaxially mounted to the housing 120. In some examples, the articulation knob 114 moves the end 136 of the insertion tube reciprocally in first and second directions (e.g., left and right). In some examples, the articulation knob 116 moves the end 136 of the insertion tube 130 reciprocally in third and fourth directions (e.g., the insertion tube moves up and down). In some examples, a locking knob 210 is provided to fix a position of the articulation knob 114 in the first and second directions. In some examples, a locking lever 214 is provided to fix a position of the articulation knob 116 in the third and fourth directions.

The articulation knob 114 includes an alignment indicator 230 such as a solid line, a dashed line, a symbol, and/or other alignment indicia. In some examples, the alignment indicator 230 is formed on or applied to the articulation knob 114 using any suitable method such as etching, painting, laser printing or etching, etc. and/or formed during molding. In some examples, the alignment indicator 230 can include projections or cavities.

The articulation knob 116 includes an alignment indicator 234 such as a solid line, a dashed line, a symbol, and/or other alignment indicia as best seen in FIG. 2B. The housing 120 includes an alignment indicator 238 such as a solid line, a dashed line, a symbol, and/or other alignment indicia. Before or during a procedure, the operator knows that the endoscopic device is in the neutral position when the alignment indicator 230 on the articulation knob 114 is aligned with the alignment indicator 234 on the articulation knob 116 and the alignment indicator 238 on the housing 120.

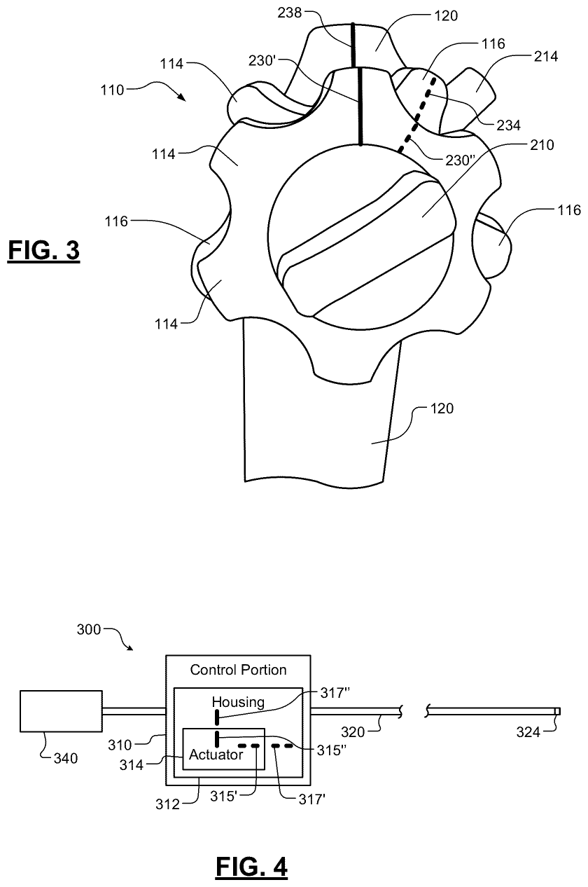

Referring now to FIG. 3, in some examples, the alignment indicator 234 on the articulation knob 116 may be in a difficult to see location. For example in FIGS. 2A and 2B, the alignment indicator 234 on the articulation knob 116 may be obscured by the articulation knob 114 when the articulation knob 114 is in the neutral position (as shown by arrow 235). In some examples, the articulation knob 114 includes additional alignment indicia. For example, the articulation knob 114 includes alignment indicators 230′ and 230″ that are rotationally offset from one another.

The articulation knob 114 is aligned in the neutral position when the alignment indicator 230′ is aligned with the alignment indicator 238 on the housing 120. Since the articulation knob 116 is located underneath the articulation knob 114 when the articulation knob 114 is aligned in the neutral position, the articulation knob 114 further includes the alignment indicator 230″ that aligns with the alignment indicator 234 on the articulation knob 116 when the articulation knob 116 is in the neutral position (after the articulation knob 114 is aligned in the neutral position relative to the housing 120). In this example, the alignment indicator 230″ and the alignment indicator 234 are rotationally offset from the alignment indicator 230′ and the alignment indicator 238 (e.g., to a position where the alignment indicators can be seen given the shape and rotational position of the articulation knobs 114 and 116). In some examples, the housing 120 includes the alignment indicator 234 instead of the articulation knob 114.

In some examples, color coding, dotted lines, solid lines, and/or other characters or patterns may be used to help the operator align the different pairs of actuators. For example in FIG. 3, the alignment indicators 230′ and 238 on the articulation knob 114 and the housing 120 are solid lines in a first color. The alignment indicators 230″ and 234 on the articulation knob 114 and the articulation knob 114 use a different pattern such as are dotted lines and/or the first color or a second color different than the first color.

Referring now to FIG. 4, an endoscopic device 300 includes a control portion 310 with a housing 312 and an actuator 314 such as an articulation lever. An insertion tube 320 extends from the housing 312 and includes an articulating end 324. The actuator 314 articulates the articulating end 324 of the insertion tube 320. The actuator 314 includes one or more alignment indicators 315′ and 315″ and the housing 312 includes one or more alignment indicators 317′ and 317″.

In some examples, the actuator 314 moves reciprocally in one direction or plane (e.g., moving the articulating end up and down or left and right). The actuator 314 includes one of the alignment indicators 315′ or 315″ that aligns with one of the alignment indicators 317′ or 317″, respectively, on the housing 312. In other examples, the actuator 314 moves in more than one direction or plane (e.g., moving the articulating end up and down and left and right) and the actuator 314 includes the alignment indicators 315′ and 315″. The housing 312 includes the alignment indicators 317′ and 317″ to allow alignment in the neutral position in two planes.

The foregoing description is merely illustrative in nature and is in no way intended to limit the disclosure, its application, or uses. The broad teachings of the disclosure can be implemented in a variety of forms. Therefore, while this disclosure includes particular examples, the true endoscopic device of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the endoscopic device of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

What is claimed is:

1. An endoscopic device comprising:
a control portion including a housing and an actuator;
an insertion tube configured for insertion into a cavity of a patient to perform at least one of visual inspection and a therapeutic intervention,
wherein the actuator is configured to articulate an articulating end of the insertion tube;
a first alignment indicator on the housing; and
a second alignment indicator on the actuator,
wherein when the second alignment indicator on the actuator is aligned with the first alignment indicator on the housing, the insertion tube is articulated in a neutral position allowing insertion and removal of the insertion tube from the cavity of the patient.

2. The endoscopic device of claim 1, wherein the actuator includes a first articulation knob and a second articulation knob that are mounted coaxially.

3. The endoscopic device of claim 2, wherein:
the first articulation knob includes the second alignment indicator, and
the second articulation knob includes a third alignment indicator.

4. The endoscopic device of claim 3, wherein the insertion tube is in the neutral position when the second alignment indicator on the first articulation knob and the third alignment indicator on the second articulation knob are aligned with the first alignment indicator on the housing.

5. The endoscopic device of claim 1, wherein:
the actuator includes a first articulation knob and a second articulation knob, and
the first articulation knob includes the second alignment indicator and a third alignment indicator, and
one of the housing and the second articulation knob includes a fourth alignment indicator.

6. The endoscopic device of claim 5, wherein the insertion tube is in the neutral position when the second alignment indicator on the first articulation knob is aligned with the first alignment indicator on the housing and the third alignment indicator on the first articulation knob is aligned with the fourth alignment indicator on the second articulation knob.

7. The endoscopic device of claim 5, wherein the first alignment indicator and the second alignment indicator are visually different than the third alignment indicator and the fourth alignment indicator.

8. The endoscopic device of claim 1, wherein the actuator comprises a lever.
